(19) United States
(12) Patent Application Publication
(10) Pub. No.: US 2018/0242605 A1
(43) Pub. Date: Aug. 30, 2018

(54) SUPERHEATED STEAM STERILIZATION APPARATUS

(71) Applicant: SATAKE CORPORATION, Tokyo (JP)

(72) Inventors: Kazunobu KAJIHARA, Tokyo (JP); Tsutomu HAKO, Tokyo (JP); Shogo SUNADA, Tokyo (JP)

(21) Appl. No.: 15/754,611

(22) PCT Filed: Aug. 22, 2016
(86) PCT No.: PCT/JP2016/074325

§ 371 Date: Feb. 23, 2018

(30) Foreign Application Priority Data
Aug. 26, 2015 (JP) 2015166400
Sep. 3, 2015 (JP) 2015173884

(51) Int. Cl. A23B 9/02; A23L 3/22; A61L 2/07

(52) U.S. Cl. CPC A23B 9/025; A23V 2002/00; A61L 2/07; A23L 3/225

(57) ABSTRACT

Provided is a superheated steam sterilization apparatus capable of continuously sterilizing a large amount of grain and of capable of eliminating uneven application of superheated steam even when a large amount of grain is loaded thereinto. A superheated steam sterilization apparatus according to one embodiment of the present invention includes: a horizontal cylindrical part including one end formed with a supply port for supplying an object to be sterilized and an opposite end formed with a discharge port for discharging a sterilized object; a rotating shaft part rotatably disposed in the horizontal cylindrical part, and including a helical screw configured to transfer the supplied object to be sterilized and an agitating member including a plurality of agitating blades configured to agitate the object to be sterilized; a plurality of superheated steam nozzles disposed above the horizontal cylindrical part; a superheated steam box disposed on the horizontal cylindrical part, divided into a plurality of sections along an axial direction, and configured to temporarily store superheated steam supplied from the superheated steam nozzles; and a steam outlet for applying superheated steam stored in the superheated steam box to the object to be sterilized which is transferred and agitated in the horizontal cylindrical part.

### TECHNICAL FIELD

The present invention relates to superheated steam sterilization apparatus for grains and, more particularly, to a superheated steam sterilization apparatus configured to sterilize brown rice obtained by hulling paddy at a stage prior to packing into a bag so that brown rice can be distributed in the hulled state without being contaminated with insect pests.

### BACKGROUND ART

Hitherto, grain sterilizing methods and apparatus of the type described above are publicly known and disclosed, for example, in Patent Literature 1. PTL 1 describes a sterilization apparatus (e.g. an ultraviolet sterilization lamp) which is attached to the discharge side of a hulling machine or a rice polishing machine. Brown rice or polished rice is sterilized by ultraviolet light from the ultraviolet sterilization lamp at a stage prior to packing into a bag. Accordingly, the eggs of insect pests cannot be hatched, and the whole rice grains are protected from being contaminated with insect pests. However, the sterilization apparatus described in PTL 1 is a simple apparatus attached to the discharge side of a hulling machine or a rice polishing machine and inferior in sterilization capability and therefore cannot be said to provide a sufficient sterilization effect.

There is known another sterilization method in which superheated steam (dry superheated air) obtained by superheating steam to 100° C. or more is used as a heating medium to sterilize grains. Such techniques are disclosed, for example, in Patent Literature 2 and Patent Literature 3.

PTL 2 describes a sterilization apparatus configured to blow superheated steam into a rotary valve. With this structure, a sufficient sterilization effect can be obtained despite a simple apparatus. However, the sterilization apparatus is not practical to treat a large amount of grains because the apparatus is configured such that grains are purposely supplied into a rotary valve so as to be sterilized with superheated steam.

PTL 3 describes a sterilization apparatus in which an agitating device is horizontally installed in a horizontal cylindrical container, and multiple common nozzles used for both superheated steam and saturated steam are provided at the upper end of the horizontal cylindrical container. The sterilization apparatus has the advantage of being surely applicable to various objects to be sterilized by effectively using latent heat. However, partition plates surrounding each common nozzle are formed in the shape of oblong plates having short vertical sides; therefore, the partition plates cannot reach the area where the agitating device is disposed. Consequently, the agitating device is placed in an open state (see FIG. 3 of PTL 3), so that the action and effect of the partition plates can hardly be exerted on the above-described area. As a result, there may occur uneven application of superheated steam in the area where the agitating device is disposed.

Regarding the maintainability of the sterilization apparatus described in PTL 3, it is only mentioned in paragraph [0013] of PTL 3 that “A drain pipe is preferably provided via an opening-closing valve at any position of the lowermost portion having a semicircular cross-section of the lower part of the horizontal cylindrical container 5”. In other words, when the interior of the horizontal cylindrical container is cleaned, or when there is a failure in the agitating device horizontally installed in the horizontal cylindrical container, a plate member covering the upper side of the horizontal cylindrical container needs to be removed to open the container. For this reason, the sterilization apparatus cannot necessarily be said to have excellent maintainability.

### CITATION LIST

### Patent Literature

PTL 1: Japanese Patent Application Laid-Open Publication No. S62-58951

PTL 2: Japanese Patent Application Laid-Open Publication No. H10-211103

PTL 3: Japanese Patent Application Laid-Open Publication No. H8-89222

### SUMMARY OF INVENTION

### Technical Problem

In view of the above-described circumstances, it is desirable to provide a superheated steam sterilization apparatus capable of continuously sterilizing a large amount of grain and of eliminating uneven application of superheated steam even if a large amount of grain is loaded thereinto.

It is also desirable to provide a superheated steam sterilization apparatus capable of continuously sterilizing a large amount of grain and superior in maintainability.

The present invention has been made to solve at least a part of the above-described problems and can be realized, for example, in the following aspects.

### Solution to Problem

A first aspect of the present invention provides a superheated steam sterilization apparatus. The superheated steam sterilization apparatus includes: a horizontal cylindrical part including one end formed with a supply port for supplying an object to be sterilized and an opposite end formed with a discharge port for discharging a sterilized object; a rotating shaft part rotatably disposed in the horizontal cylindrical part, and including a helical screw configured to transfer the supplied object to be sterilized and an agitating member including a plurality of agitating blades configured to agitate the object to be sterilized; a plurality of superheated steam nozzles disposed above the horizontal cylindrical part; a superheated steam box disposed on the horizontal cylindrical part, divided into a plurality of sections along an axial direction, and configured to temporarily store superheated steam supplied from the superheated steam nozzles; and a steam outlet for applying the superheated steam stored in the superheated steam box to the object to be sterilized which is transferred and agitated in the horizontal cylindrical part.

According to the first aspect, a large amount of object (grain) to be sterilized can be continuously sterilized by the horizontal cylindrical part and the rotating shaft part. In addition, superheated steam from the superheated steam nozzles can be temporarily stored in the superheated steam box and then applied to the object to be sterilized from the steam outlet set to an appropriate degree of opening. Therefore, the object to be sterilized can be sterilized by superheated steam applied over a widened area (the steam outlet has an opening area larger than that of the superheated steam nozzle having a small nozzle diameter). Accordingly, superheated steam is applied not locally but to a wide area even when a large amount of object (grain) to be sterilized is loaded. Consequently, it is possible to eliminate uneven application of superheated steam to the object to be sterilized.

According to a second aspect of the present invention, in the first aspect, the superheated steam sterilization apparatus further includes an opening-closing shutter provided at the steam outlet. The opening-closing shutter is configured to adjust the amount of the superheated steam to be applied by adjusting the opening area of the steam outlet.

According to the second aspect, if the degree of opening of the opening-closing shutter is set to fully open, it is also possible to sterilize grains containing a large number of bacteria (e.g. grains that seem to have a large number of bacteria grown thereon, for example, brown rice having been stored for a long period of time in a state of being mixed with paddy and chaff). On the other hand, if the degree of opening of the opening-closing shutter is set to half-open, it is also possible to sterilize grains containing a small number of bacteria (e.g. polished and sorted grains obtained by completely removing paddy and chaff from brown rice) in an energy-conserving manner with a reduced supply of the superheated steam.

According to a third aspect of the present invention, in the first or second aspect, the superheated steam sterilization apparatus further includes a cooling part connected to the discharge port of the horizontal cylindrical part for discharging the sterilized object. The cooling part includes a horizontally disposed screen tube, a helical screw and an agitating member which are disposed in the screen tube, and a suction fan configured to send cooling air into the screen tube.

According to the third aspect, the cooling part is connected to the discharge port of the horizontal cylindrical part for discharging the sterilized object. Therefore, in the cooling part, the sterilized object, which has been sterilized by the superheated steam, is supplied into the screen tube. In addition, the cooling part carries out agitating with the agitating member while taking the outside air into the screen tube from an outside air inlet by driving the suction fan. Thus, effective cooling processing is performed. Meanwhile, foreign substances, gravel, etc. caught between the grains of the object to be sterilized drop through meshes of the screen tube. Therefore, these foreign substances and gravel can be discharged to the outside of the machine.

A fourth aspect of the present invention provides a superheated steam sterilization apparatus. The superheated steam sterilization apparatus includes: a horizontal cylindrical part including one end formed with a supply port for supplying an object to be sterilized and an opposite end formed with a discharge port for discharging a sterilized object; a rotating shaft part rotatably disposed in the horizontal cylindrical part, and configured to transfer the supplied object to be sterilized while agitating the object to be sterilized; a superheated steam nozzle disposed above the horizontal cylindrical part; a superheated steam box disposed between the superheated steam nozzle and the horizontal cylindrical part, and configured to temporarily store superheated steam supplied from the superheated steam nozzle; and a steam outlet for applying the superheated steam stored in the superheated steam box to the object to be sterilized which is transferred and agitated in the horizontal cylindrical part.

The forth aspect offers advantages similar to those of the first aspect.

A fifth aspect of the present invention provides a superheated steam sterilization apparatus. The superheated steam sterilization apparatus includes: a horizontal cylindrical part including one end formed with a supply port for supplying an object to be sterilized and an opposite end formed with a discharge port for discharging a sterilized object; a rotating shaft part rotatably disposed in the horizontal cylindrical part, and including a helical screw configured to transfer the supplied object to be sterilized and an agitating member including a plurality of agitating blades configured to agitate the object to be sterilized; a plurality of superheated steam nozzles disposed above the horizontal cylindrical part; a superheated steam box disposed on the horizontal cylindrical part, divided into a plurality of sections along an axial direction, and configured to temporarily store superheated steam supplied from the superheated steam nozzles; and a steam outlet for applying the superheated steam stored in the superheated steam box to the object to be sterilized which is transferred and agitated in the horizontal cylindrical part. The horizontal cylindrical part is formed to be splittable into a base portion arcuately covering most part of the rotating shaft part and a maintenance cover portion removable from the base portion and arcuately covering a part of the rotating shaft part.

The fifth aspect offers advantages similar to those of the first aspect. Moreover, when the interior of the horizontal cylindrical part is cleaned, or when the helical screw and the agitating member are repaired, the interior of the horizontal cylindrical part can be opened simply by removing the maintenance cover portion, which is exclusively used for maintenance, from the base portion, and thus the rotating shaft part is exposed. Accordingly, very excellent maintainability is obtained.

According to a sixth aspect of the present invention, in the fifth aspect, the base portion of the horizontal cylindrical part is formed in the shape of a circular are as viewed in a cross section. The circular are has a central angle exceeding 180° and extending from the uppermost point of a circle formed by the outline of the horizontal cylindrical part to the lowermost point of the circle and further extending upward from the lowermost point so that the base portion receives drain water generated from the superheated steam after sterilization.

According to the sixth aspect, the base portion is not in the shape of a semicircle having a central angle of 180° and extending from the uppermost point to the lowermost point of the circle, but in the shape of the circular arc having the central angle exceeding 180° and further extending upward from the lowermost point. Therefore, there is no fear that drain water produced in the cylindrical part may drop when the horizontal cylindrical part is disassembled. In other words, drain water is surely received by the circular arc-shaped portion, so that there is no fear of drain water dropping or spilling out over the floor when the apparatus is disassembled or cleaned.

According to a seventh aspect of the present invention, in the fifth or sixth aspect, the maintenance cover portion of the horizontal cylindrical part includes a plurality of handles for holding the maintenance cover portion at the time of maintenance.

According to the seventh aspect, the operator can remove and attach the maintenance cover portion by holding the handles at the time of maintenance. Accordingly, it is easy to remove and attach the maintenance cover portion, and operability is also improved.

An eighth aspect of the present invention provides a superheated steam sterilization apparatus. The superheated steam sterilization apparatus includes: a horizontal cylindrical part including one end formed with a supply port for supplying an object to be sterilized and an opposite end formed with a discharge port for discharging a sterilized object; a rotating shaft part rotatably disposed in the horizontal cylindrical part, and configured to transfer the supplied object to be sterilized while agitating the object to be sterilized; a superheated steam nozzle disposed above the horizontal cylindrical part; a superheated steam box disposed between the superheated steam nozzle and the horizontal cylindrical part, and configured to temporarily store superheated steam supplied from the superheated steam nozzle; and a steam outlet for applying the superheated steam stored in the superheated steam box to the object to be sterilized which is transferred and agitated in the horizontal cylindrical part. The horizontal cylindrical part is formed to be splittable into a base portion partially covering the rotating shaft part and a maintenance cover portion removable from the base portion and partially covering the rotating shaft part.

According to a ninth aspect of the present invention, the base portion of the horizontal cylindrical part has, as viewed in a cross section, a lowermost portion of the horizontal cylindrical part and a portion extending upward from the lowermost portion.

The eighth and ninth aspects offer advantages similar to those of the fifth and sixth aspects.

### BRIEF DESCRIPTION OF DRAWINGS

FIG. 1 is a vertical sectional view schematically showing the structure of a superheated steam sterilization apparatus according to the present invention.

FIG. 2 is a schematic perspective view of a horizontal cylindrical part with a cover portion removed therefrom.

FIG. 3 is a vertical sectional view of the superheated steam sterilization apparatus, taken along the line A-A in FIG. 1.

FIG. 4 is an enlarged perspective view showing a superheated steam box of the superheated steam sterilization apparatus.

FIG. 5 is a sectional view of the superheated steam sterilization apparatus shown in FIG. 3, with the cover portion removed therefrom.

### DESCRIPTION OF EMBODIMENTS

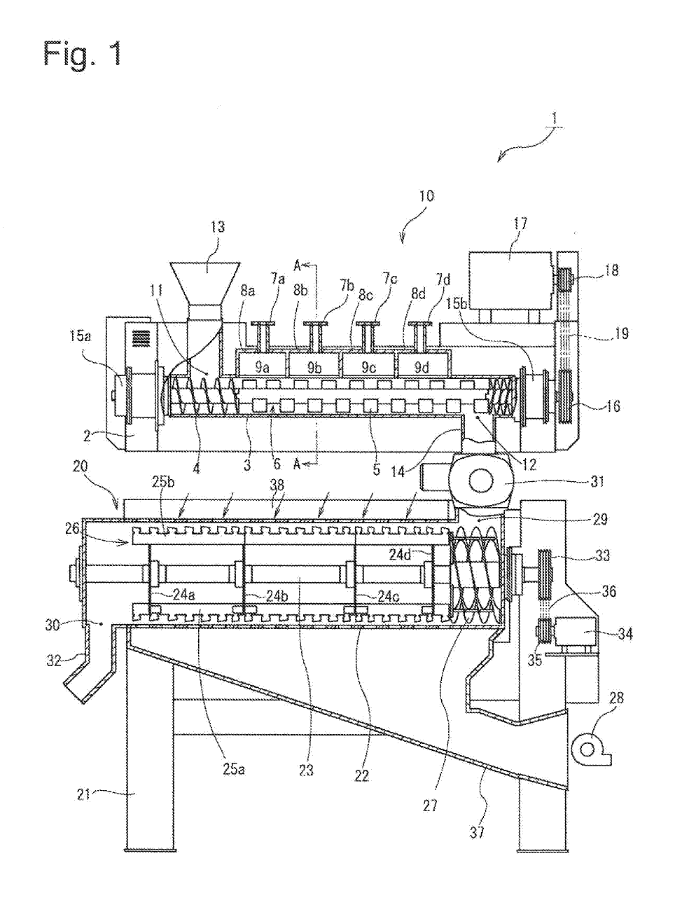

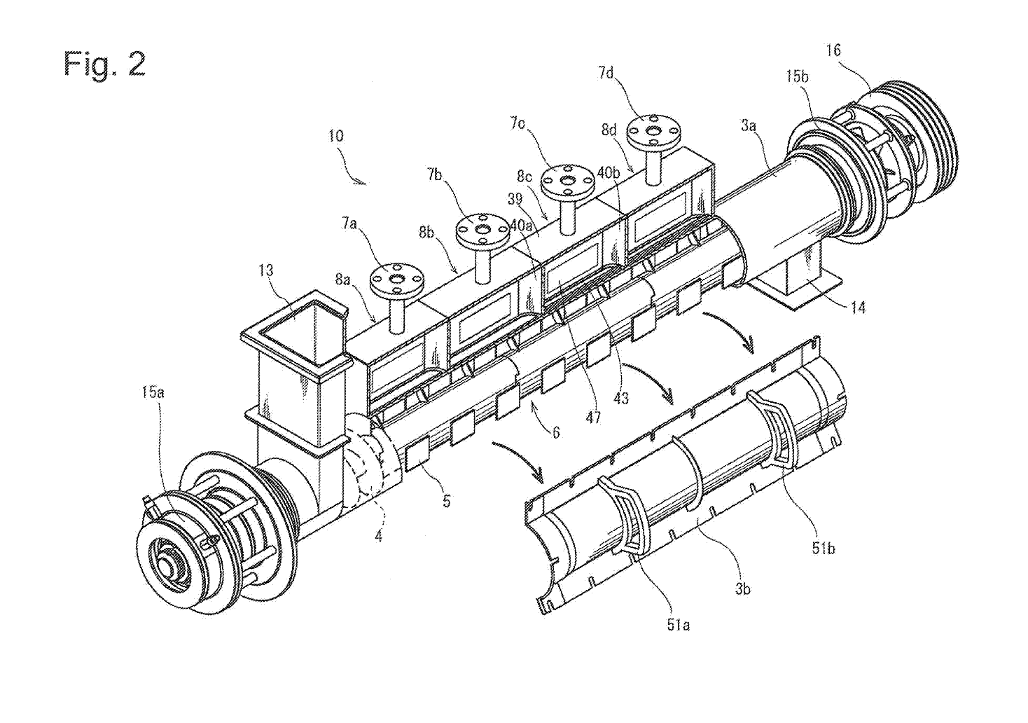

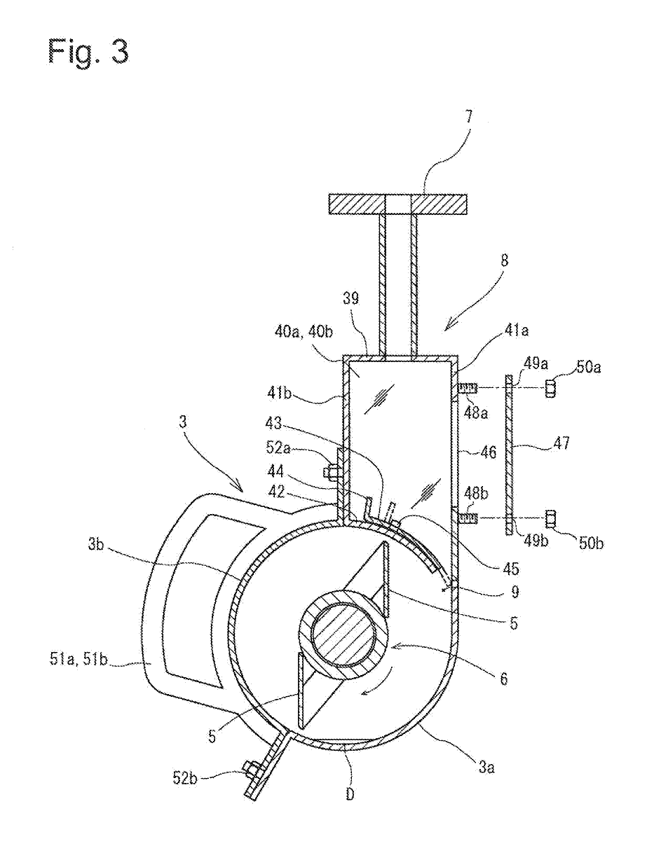

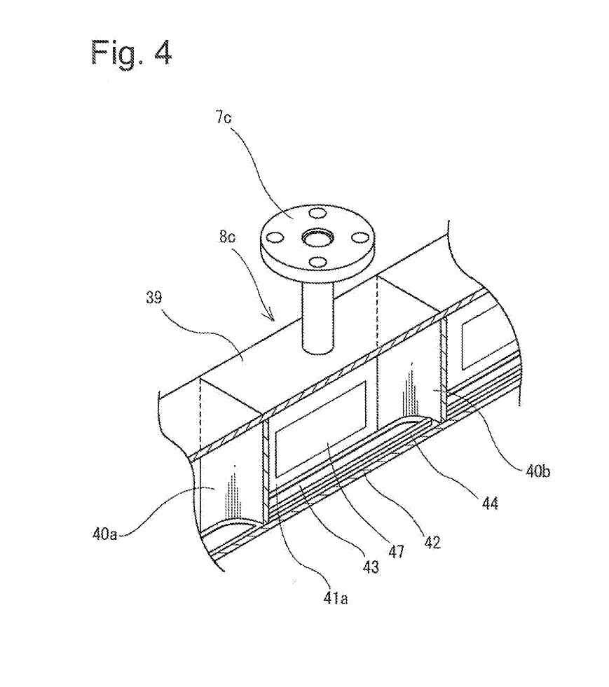

A preferred embodiment of the present invention will be explained below with reference to the drawings. FIG. 1 is a vertical sectional view schematically showing the structure of a superheated steam sterilization apparatus 1 according to one embodiment of the present invention. FIG. 2 is a schematic perspective view of a horizontal cylindrical part 3 (hereinafter occasionally referred to simply as “the cylindrical part 3”) of the superheated steam sterilization apparatus 1 with a cover portion 3b removed therefrom. FIG. 3 is a vertical sectional view of the superheated steam sterilization apparatus 1, taken along the line A-A in FIG. 1. FIG. 4 is an enlarged perspective view showing a superheated steam box 8 of the superheated steam sterilization apparatus 1.

As shown in FIG. 1, the superheated steam sterilization apparatus 1 according to one embodiment of the present invention includes a superheated sterilization part 10. The superheated sterilization part 10 includes a horizontal cylindrical part 3, a rotating shaft part 6, multiple (four in this embodiment) superheated steam nozzles 7a to 7d (superheated steam nozzles 7a to 7d will occasionally be referred to collectively as “the superheated steam nozzle 7”), the same number of superheated steam boxes 8a to 8d as the number of superheated steam nozzles 7 (superheated steam boxes 8a to 8d will occasionally be referred to collectively as “the superheated steam box 8”), and the same number of steam outlets 9a to 9d as the number of superheated steam nozzles 7 (steam outlets 9a to 9d will occasionally be referred to collectively as “the steam outlet 9”). The horizontal cylindrical part 3 is horizontally installed in a machine frame 2. The rotating shaft part 6 includes a helical screw 4 and an agitating member 5 which are provided on a shaft extending horizontally. The helical screw 4 is rotatably disposed in the horizontal cylindrical part 3, and transfers grains supplied into the horizontal cylindrical part 3. The agitating member 5 includes multiple agitating blades that agitates the grains. The superheated steam nozzle 7 is installed above the horizontal cylindrical part 3. The superheated steam box 8 is disposed on the cylindrical part 3. The superheated steam box 8 is divided into a plurality of sections (superheated steam boxes 8a to 8d) along the axial direction. The superheated steam box 8 temporarily stores superheated steam in a distributed manner which is supplied from the superheated steam nozzle 7. The steam outlet 9 is configured to apply the superheated steam from the superheated steam box 8 to grains which are being transferred and agitated in the horizontal cylindrical part 3.

The horizontal cylindrical part 3 includes a grain supply port 11 formed in an upper portion thereof at one end side in the axial direction. The cylindrical part 3 includes a grain discharge port 12 formed in a lower portion thereof at the other end side in the axial direction. A raw grain supply chute 13 is connected to the supply port 11, and a grain discharge chute 14 is connected to the discharge port 12. The discharge chute 14 communicates with a grain cooling part 20 in a subsequent step through a rotary valve or the like.

The rotating shaft part 6 is supported by a bearing 15a at one end side in the axial direction thereof. The other end side in the axial direction of the rotating shaft part 6 is supported by a bearing 15b. In addition, a drive pulley 16 that rotates the rotating shaft part 6 is installed at the other end side of the rotating shaft part 6. A rotational force of a drive motor 17 is transmitted to the drive pulley 16 through a motor pulley 18 and a belt 19.

The superheated steam sterilization apparatus 1 further includes a grain cooling part 20 in a stage following the superheated sterilization part 10. The grain cooling part 20 includes, as shown in FIG. 1, a screen tube 22, a rotating shaft 23, an agitating member 26, a helical screw 27, and a suction fan 28. The screen tube 22 is horizontally installed on a frame 21 and has multiple slits. The rotating shaft 23 is disposed in the screen tube 22. The agitating member 26 includes a plurality of arm members 24a to 24d radially attached to the rotating shaft 23 at a plurality of positions of the rotating shaft 23 (arm members 24a to 24d will occasionally be referred to collectively as “the arm member 24”). The agitating member 26 further includes elongate agitating blades 25a and 25b supported by the arm members 24. The helical screw 27 is attached to the rotating shaft 23, and transfers grains. The suction fan 28 sends cooling air into the screen tube 22. The screen tube 22 has a grain supply port 29 formed in an upper portion thereof at one end side in the axial direction and further has a grain discharge port 30 formed in a lower portion thereof at the other end side in the axial direction. The discharge chute 14 of the superheated sterilization part 10 is connected to the supply port 29 through a rotary valve 31. A discharge chute 32 that discharges cooled grains to the outside of the machine is connected to the discharge port 30.

A drive pulley 33 that rotates the rotating shaft 23 is installed at one end side of the rotating shaft 23. A rotational force of a drive motor 34 is transmitted to the drive pulley 33 through a motor pulley 35 and a belt 36. In the grain cooling part 20, grains superheated-sterilized in the superheated sterilization part 10 are supplied into the screen tube 22 to cool down the heated grains. The grain cooling part 20 performs agitating with the agitating member 26 while taking the outside air into the screen tube 22 from an outside air inlet 38 by driving the suction fan 28. Consequently, heat accumulated between the grains is taken away therefrom, and thus cooling is performed. In addition, foreign substances, gravel, etc. caught between the grains drop through the meshes of the screen tube 22. Accordingly, it is also possible to separate foreign substances, gravel, etc. from the grains and to discharge the separated substances to the outside of the machine. A foreign substance collecting trough 37 is installed underneath the screen tube 22. The foreign substance collecting trough 37 collects foreign substances, gravel, etc. separated from the grains and discharges the collected substances to the outside of the machine.

Referring to FIG. 2, the structure of the superheated steam box 8 will be explained in detail. The superheated steam box 8 is provided on a base portion 3a of the horizontal cylindrical part 3 so as to partially cover the top of the horizontal cylindrical part 3. As shown in FIGS. 1,2, 3 and 4, the superheated steam box 8 is divided into a plurality of superheated steam boxes 8a to 8d along the axial direction. These superheated steam boxes 8a to 8d are continuously disposed along the axial direction. The superheated steam boxes 8a to 8d each store superheated steam. The superheated steam boxes 8a to 8d are each formed in the shape of a box by being surrounded with a top wall 39, a plurality of axially spaced partition walls 40a and 40b, sidewalls 41a and 41b, and an arcuate bottom wall 42. The top wall 39 has holes in communication with the steam nozzle 7. The arcuate bottom wall 42 is formed with a steam outlet 9. With this structure, the superheated steam box 8 can apply superheated steam to grains in the cylindrical part 3. In addition, the bottom wall 42 is provided with an opening-closing shutter 43. The opening-closing shutter 43 can adjust the amount of superheated steam to be applied into the cylindrical part 3 by adjusting the opening area of the steam outlet 9. More specifically, the opening-closing shutter 43 is configured such that the opening area of the steam outlet 9 can be adjusted by the operator holding an adjustment knob 44 provided for the opening-closing shutter 43 in his or her hand and sliding the adjustment knob 44 along the bottom wall 42 (the one-dot chain line in FIG. 3 shows the steam outlet 9 in a closed position). It should be noted that the reference sign 45 denotes a stopper for the adjustment knob 44, and that the stopper is formed to project from the bottom wall 42.

The sidewall 41a has an inspection opening 46 formed therein so that the operator can adjust the opening-closing shutter 43. In addition, the sidewall 41a is provided with an inspection lid 47 for closing the inspection opening 46 (see FIG. 3). The sidewall 41a is provided with bolts 48a and 48b projecting therefrom. The inspection opening 46 can be closed with the inspection lid 47 by fitting the inspection lid 47 onto the bolts 48a and 48b in such a manner that the bolts 48a and 48b are inserted into mounting holes 49a and 49b in the inspection lid 47 and then fastening the inspection lid 47 with nuts 50a and 50b.

Next, the maintenance structure of the horizontal cylindrical part 3 will be explained in detail with reference to FIGS. 2 and 3. The horizontal cylindrical part 3 includes a cylindrical wall configured to be splittable into a base portion 3a and a maintenance cover portion 3b (hereinafter occasionally referred to simply as “the cover portion 3b”). The base portion 3a arcuately covers most part of the rotating shaft part 6, which includes the helical screw 4 and the agitating member 5. The maintenance cover portion 3b arcuately covers a part of the rotating shaft part 6. The maintenance cover portion 3b is removable from the base portion 3a.

Referring to FIG. 3, the base portion 3a is formed in the shape of a circular arc (circular arc including the lowermost portion of the horizontal cylindrical part 3) extending, in terms of positions on the clock face, beyond the 6 o'clock position to the 7 or 8 o'clock position. The reason for this is as follows. If the terminating end of the base portion 3a ends off at the 6 o'clock position on the clock, drain water D (reference sign D in FIG. 3) produced in the cylindrical part 3 drops when the cylindrical part 3 is disassembled. By forming the base portion 3a in the shape of a circular arc extending beyond the 6 o'clock position to the 7 or 8 o'clock position, the drain water D is received in the arcuate portion as shown in FIG. 3; therefore, there is no fear of drain water D dropping when the cylindrical part 3 is disassembled. On the other hand, the maintenance cover portion 3b is formed in the shape of a circular arc extending, in terms of positions on the clock face, from the 7 or 8 o'clock position to the 12 o'clock position (i.e. a circular arc whose central angle is not in excess of 180°; in other words, a circular are not including the lowermost portion of the horizontal cylindrical part 3). Thus, the maintenance cover portion 3b can be reduced in size and becomes easy for the operator to handle.

In addition, the maintenance cover portion 3b is provided with handles 51a and 51b for removal. Therefore, when maintenance is performed, the operator can remove fixing nuts 52a to 52b while holding the handles 51a and 51b, thereby enabling the maintenance cover portion 3b to be removed easily. Accordingly, removal operability is also improved (see FIG. 2).

The following is an explanation of the operation of the superheated steam sterilization apparatus configured as stated above. First, the inspection lid 47 shown in FIG. 3 is opened, and the degree of opening of the steam outlet 9 in the superheated steam box 8 is adjusted in advance so as to conform to conditions such as the temperature for sterilizing grains which are objects to be sterilized. In addition, the rotational speed of the rotating shaft part 6 is set appropriately to adjust conditions such as the sterilization processing time.

Next, grains are supplied from the supply chute 13 (see FIG. 1). Consequently, the grains are horizontally transferred continuously by the helical screw 4 and the agitating member 5 in the horizontal cylindrical part 3. At this time, superheated steam supplied from the superheated steam nozzle 7 is stored in the superheated steam box 8 and applied to the grains from the steam outlet 9 set to an appropriate degree of opening. The superheated steam is superheated steam (dry superheated air) obtained by superheating steam to 100° C. or more, and has a temperature range of from 200° C. to 400° C., for example.

If superheated steam is applied to grains directly from the superheated steam nozzle 7, the grains are sterilized only locally (at points) because superheated steam is applied from the superheated steam nozzle 7 having a small nozzle diameter. In contrast to this, when superheated steam supplied from the superheated steam nozzle 7 is temporarily stored in the superheated steam box 8 and thereafter applied from the steam outlet 9 set to an appropriate degree of opening, the grains are sterilized over a wide surface area (the steam outlet 9 has an opening area larger than that of the superheated steam nozzle 7 having a small nozzle diameter). Accordingly, it is possible to eliminate uneven application of superheated steam.

In addition, the degree of opening of the steam outlet 9 of each of the superheated steam boxes 8a to 8d can be adjusted by the opening-closing shutter 43. Therefore, if the degree of opening of the steam outlet 9 is set to fully open, it is also possible to sterilize grains containing a large number of bacteria (e.g. grains that seem to have a large number of bacteria grown thereon, for example, brown rice having been stored for a long period of time in a state of being mixed with paddy and chaff; chaff cannot be said to be germ-free but contains some microorganisms and bacteria). On the other hand, if the degree of opening is set to half-open, it is also possible to sterilize grains containing a small number of bacteria (e.g. polished and sorted grains obtained by completely removing paddy and chaff from brown rice; and relatively clean grains from which chaff has been removed) in an energy-conserving manner with a reduced supply of superheated steam.

Next, grains having passed through the area where the superheated steam boxes 8a to 8d are disposed are continuously discharged from the discharge port 12 while being agitating by the agitating member 5. The grains discharged from the discharge port 12 are supplied to the supply port 29 of the grain cooling part 20 through the rotary valve 31 (FIG. 1). In the grain cooling part 20, the grains sterilized by superheated steam are supplied into the screen tube 22. The grain cooling part 20 carries out agitating with the agitating member 26 while taking the outside air into the screen tube 22 from the outside air inlet 38 by driving the suction fan 28. Thus, the grains are cooled. Foreign substances, gravel, etc. caught between the grains drop through the meshes of the screen tube 22 and are discharged to the outside of the machine via the foreign substance collecting trough 37. The grains, after having undergone cooling, pass through the discharge port 30 and are discharged to the outside of the machine from the discharge chute 32.

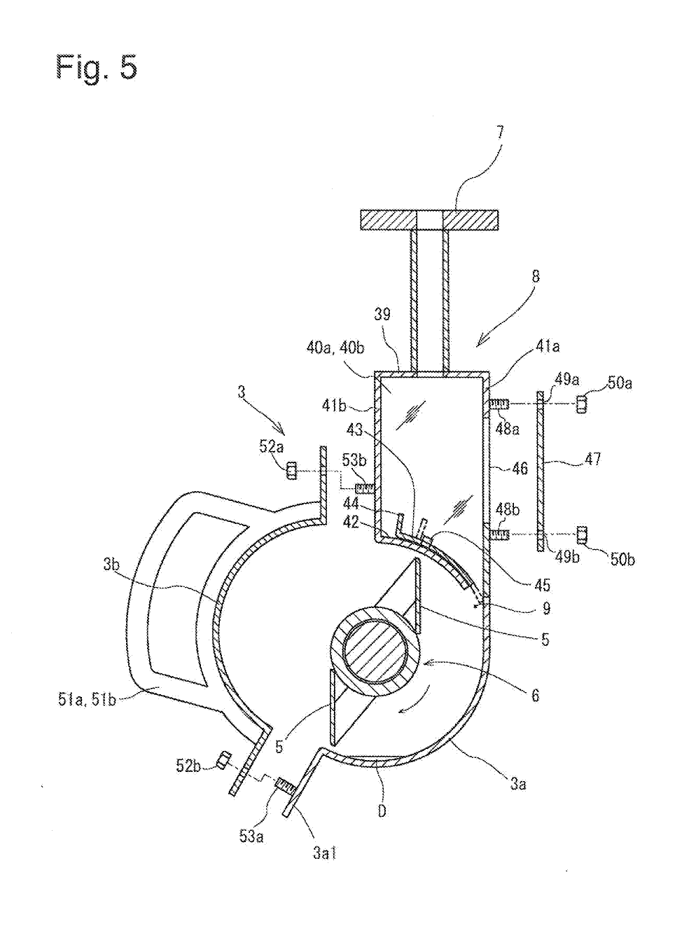

When maintenance of the horizontal cylindrical part 3 is performed, first, nuts 52a and 52b are unfastened and removed respectively from a bolt 53a provided to project from a flange portion 3a1 of the base portion 3a of the horizontal cylindrical part 3 and a bolt 53b provided to project from the sidewall 41a of the superheated steam box 8. Thus, the operator can remove the maintenance cover portion 3b by holding the handles 51a and 51b attached thereto (see FIG. 5).

As has been stated above, according to this embodiment, the superheated steam sterilization apparatus 1 includes: the horizontal cylindrical part 3 including the one end formed with the supply port 11 for supplying an object to be sterilized and the other end formed with the discharge port 12 for discharging a sterilized object; the rotating shaft part 6 rotatably disposed in the horizontal cylindrical part 3, and including the helical screw 4 configured to transfer the supplied object to be sterilized and the agitating member 5 including the plurality of agitating blades and configured to agitate the object to be sterilized; the plurality of superheated steam nozzles 7 disposed above the horizontal cylindrical part 3; the superheated steam box 8 disposed on the horizontal cylindrical part 3, divided into the plurality of sections along the axial direction, and configured to temporarily store superheated steam supplied from the superheated steam nozzles 7; and the steam outlet 9 for applying the superheated steam stored in the superheated steam box 8 to the object to be sterilized which is transferred and agitated in the horizontal cylindrical part 3. Accordingly, superheated steam from the superheated steam nozzles 7 can be temporarily stored in the superheated steam box 8 and then applied to the object to be sterilized from the steam outlet 9 set to an appropriate degree of opening. Consequently, the object to be sterilized can be sterilized by superheated steam applied over a widened area (the steam outlet 9 has an opening area larger than that of the superheated steam nozzle 7 having a small nozzle diameter). Because superheated steam is applied not locally but to a wide area, it is possible to eliminate uneven application of superheated steam to the object to be sterilized.

In addition, the horizontal cylindrical part 3 is formed to be splittable into the base portion 3a arcuately covering most part of the rotating shaft part 6 and the maintenance cover portion 3b removable from the base portion 3a and arcuately covering a part of the rotating shaft part 6. Accordingly, when the interior of the horizontal cylindrical part 3 is cleaned, or when the helical screw 4 and the agitating member 5 are repaired, the interior of the horizontal cylindrical part 3 can be opened simply by removing the maintenance cover portion 3b, which is exclusively used for maintenance, from the base portion 3a, and thus the rotating shaft part 6 is exposed. Consequently, very excellent maintainability is obtained.

Although some embodiments of the present invention have been described above, the described embodiments of the present invention are for the purpose of facilitating the understanding of the present invention and are not intended to limit the present invention. The present invention may, as a matter of course, be modified and improved without departing from the spirit thereof, and the present invention includes equivalents thereof. In addition, the structural elements described in the claims and the specification can be arbitrarily combined or omitted within a range in which the above-mentioned problems are at least partially solved, or within a range in which at least a part of the above-mentioned advantages is achieved. For example, the number of superheated steam nozzles 7 may be one. In this case, the arrangement may be such that one intermediate chamber is provided between the superheated steam nozzle 7 and the superheated steam boxes 8a to 8d, and that superheated steam introduced into the intermediate chamber from the superheated steam nozzle 7 is further introduced into the superheated steam boxes 8a to 8d through a plurality of holes formed in the intermediate chamber. Further, the superheated steam box 8 may be a single chamber having a plurality of steam outlets 9a to 9d.

### INDUSTRIAL APPLICABILITY

The present invention is applicable to the field of superheated steam sterilization apparatus capable of continuously sterilizing a large amount of grain. For example, the present invention is also applicable as a sterilization apparatus (disinfection apparatus) for sterilizing paddy rice seeds (seed rice) against contagious diseases (rice blast, fool seedling disease, etc.) in addition to a sterilization apparatus for sterilizing brown rice obtained by hulling paddy before distribution.

### REFERENCE SIGNS LIST

1 . . . superheated steam sterilization apparatus
2 . . . machine frame
3 . . . horizontal cylindrical part
3a . . . base portion
3b . . . maintenance cover portion
3a1 . . . flange portion
4 . . . helical screw
5 . . . agitating member
6 . . . rotating shaft part
7a to 7d . . . superheated steam nozzles
8a to 8d . . . superheated steam boxes
9a to 9d . . . steam outlets
10 . . . superheated sterilization part
11 . . . supply port
12 . . . discharge port
13 . . . supply chute
14 . . . discharge chute
15a, 15b . . . bearings
16 . . . drive pulley
17 . . . drive motor
18 . . . motor pulley
19 . . . belt
20 . . . grain cooling part
21 . . . frame
22 . . . screen tube
23 . . . rotating shaft
24a to 24d . . . arm members
25a to 25d . . . elongate agitating blades
26 . . . agitating member
27 . . . helical screw
28 . . . suction fan
29 . . . supply port
30 . . . discharge port
31 . . . rotary valve
32 . . . discharge chute
33 . . . drive pulley
34 . . . drive motor
35 . . . motor pulley
36 . . . belt
37 . . . foreign substance collecting trough
38 . . . outside air inlet
39 . . . top wall
40a, 40b . . . partition walls
41a, 41b . . . side walls
42 . . . bottom wall
43 . . . opening-closing shutter
44 . . . adjustment knob
46 . . . inspection opening
47 . . . inspection lid
48a, 48b . . . bolts
49a, 49b . . . holes
50a, 50b . . . nuts
51a, 51b . . . handles
52a, 52b . . . fixing nuts
53a, 53b . . . bolts
D . . . drain water

1. A superheated steam sterilization apparatus comprising:
a horizontal cylindrical part including one end formed with a supply port for supplying an object to be sterilized and an opposite end formed with a discharge port for discharging a sterilized object;
a rotating shaft part rotatably disposed in the horizontal cylindrical part, and including a helical screw configured to transfer the supplied object to be sterilized and an agitating member including a plurality of agitating blades configured to agitate the object to be sterilized;
a plurality of superheated steam nozzles disposed above the horizontal cylindrical part;
a superheated steam box disposed on the horizontal cylindrical part, divided into a plurality of sections along an axial direction, and configured to temporarily store superheated steam supplied from the superheated steam nozzles; and
a steam outlet for applying the superheated steam stored in the superheated steam box to the object to be sterilized which is transferred and agitated in the horizontal cylindrical part.

2. The superheated steam sterilization apparatus of claim 1, further comprising:
an opening-closing shutter provided at the steam outlet, and configured to adjust an amount of the superheated steam to be applied by adjusting an opening area of the steam outlet.

3. The superheated steam sterilization apparatus of claim 1, further comprising:
a cooling part connected to the discharge port of the horizontal cylindrical part for discharging the sterilized object;
wherein the cooling part includes a horizontally disposed screen tube, a helical screw and an agitating member which are disposed in the screen tube, and a suction fan configured to send cooling air into the screen tube.

4. A superheated steam sterilization apparatus comprising:
a horizontal cylindrical part including one end formed with a supply port for supplying an object to be sterilized and an opposite end formed with a discharge port for discharging a sterilized object;
a rotating shaft part rotatably disposed in the horizontal cylindrical part, and configured to transfer the supplied object to be sterilized while agitating the object to be sterilized;
a superheated steam nozzle disposed above the horizontal cylindrical part;
a superheated steam box disposed between the superheated steam nozzle and the horizontal cylindrical part, and configured to temporarily store superheated steam supplied from the superheated steam nozzle; and
a steam outlet for applying the superheated steam stored in the superheated steam box to the object to be sterilized which is transferred and agitated in the horizontal cylindrical part.

5. A superheated steam sterilization apparatus comprising:
a horizontal cylindrical part including one end formed with a supply port for supplying an object to be sterilized and an opposite end formed with a discharge port for discharging a sterilized object;
a rotating shaft part rotatably disposed in the horizontal cylindrical part, including a helical screw configured to transfer the supplied object to be sterilized and an agitating member including a plurality of agitating blades configured to agitate the object to be sterilized;
a plurality of superheated steam nozzles disposed above the horizontal cylindrical part;
a superheated steam box disposed on the horizontal cylindrical part, divided into a plurality of sections along an axial direction, and configured to temporarily store superheated steam supplied from the superheated steam nozzles; and
a steam outlet for applying the superheated steam stored in the superheated steam box to the object to be sterilized which is transferred and agitated in the horizontal cylindrical part;
wherein the horizontal cylindrical part is formed to be splittable into a base portion arcuately covering most part of the rotating shaft part and a maintenance cover portion removable from the base portion and arcuately covering a part of the rotating shaft part.

6. The superheated steam sterilization apparatus of claim 5, wherein the base portion of the horizontal cylindrical part is formed in a shape of a circular arc as viewed in a cross section, the circular arc having a central angle exceeding 180° and extending from an uppermost point of a circle formed by an outline of the horizontal cylindrical part to a lowermost point of the circle and further extending upward from the lowermost point so that the base portion receives drain water generated from the superheated steam after sterilization.

7. The superheated steam sterilization apparatus of claim 5, wherein the maintenance cover portion of the horizontal cylindrical part includes a plurality of handles for holding the maintenance cover portion at time of maintenance.

8. A superheated steam sterilization apparatus comprising:
a horizontal cylindrical part including one end formed with a supply port for supplying an object to be sterilized and an opposite end formed with a discharge port for discharging a sterilized object;
a rotating shaft part rotatably disposed in the horizontal cylindrical part, and configured to transfer the supplied object to be sterilized while agitating the object to be sterilized;
a superheated steam nozzle disposed above the horizontal cylindrical part;
a superheated steam box disposed between the superheated steam nozzle and the horizontal cylindrical part, and configured to temporarily store superheated steam supplied from the superheated steam nozzle; and
a steam outlet for applying the superheated steam stored in the superheated steam box to the object to be sterilized which is transferred and agitated in the horizontal cylindrical part;
wherein the horizontal cylindrical part is formed to be splittable into a base portion partially covering the rotating shaft part and a maintenance cover portion removable from the base portion and partially covering the rotating shaft part.

9. The superheated steam sterilization apparatus of claim 8, wherein the base portion of the horizontal cylindrical part includes, as viewed in a cross section, a lowermost portion of the horizontal cylindrical part and a portion extending upward from the lowermost portion.
